(19) United States
(12) Patent Application Publication
(10) Pub. No.: US 2016/0001042 A1
(43) Pub. Date: Jan. 7, 2016

(54) Hybrid Lateral Vein Introducer

(71) Applicant: PRESSURE PRODUCTS MEDICAL SUPPLIES INC., Santa Barbara, CA (US)

(72) Inventors: Seth Worley, Lancaster, PA (US); Andrew Armour, Swarthmore, PA (US); Paul Kurth, Santa Barbara, CA (US)

(73) Assignee: Pressure Products Medical Supplies Inc., Santa Barbara, CA (US)

(21) Appl. No.: 14/769,406

(22) PCT Filed: Mar. 14, 2014
(86) PCT No.: PCT/US2014/029382

§ 371 Date: Aug. 20, 2015

Related U.S. Application Data
(60) Provisional application No. 61/783,527, filed on Mar. 14, 2013.

(51) Int. Cl. A61M 25/01; A61M 39/06

(52) U.S. Cl. CPC A61M 25/01; A61M 39/06; A61M 2025/0188

(57) ABSTRACT

A hybrid lateral vein introducer which includes a conventional splittable hemostatic valve, which comprises two symmetrical wing extending from a splittable hub. A conventional splittable proximal sheath portion is coupled to and extends from the hub. The sheath portion is a conventional soft pliable sheath, which has limited torqueability, but adequate flexibility to be maneuvered in the cardiac vascular system. At a predetermined distance from the valve, the sheath is coupled to a reinforced sheath portion. Reinforced sheath portion is reinforced sufficiently to allow for good integrity and crush resistance while maintaining flexibility of the sheath portion. A proximal segment of the sheath portion is reinforced, but still splittable. Alternatively, the entire length of the sheath portion including the proximal segment and distal portions are rendered splittable.

### RELATED APPLICATIONS

The present application is related to U.S. Provisional Patent Application, Ser. No. 61/783,527, filed on Mar. 14, 2013, which is incorporated herein by reference and to which priority is claimed.

### BACKGROUND

1. Field of the Invention

The invention relates to sheaths and introducers, which are utilized in a human heart, which has been altered by heart disease. More particularly, the invention relates to a hybrid splittable introducer for lateral vein cannulation.

2. Description of the Prior Art

The coronary sinus is the largest cardiac vein and serves as a conduit for access to various locations within the heart. Depending on the depth of insertion of the medical device into the coronary sinus, both the left and right atria and the left and right ventricles of the heart can be accessed and analyzed. However, introduction of a medical device into the ostium of the coronary sinus is quite difficult as a result of the structure of the heart, the difficulty in locating the coronary sinus using conventional medical technology and the constantly changing shape of the heart while beating as well as the filtered anatomy of the heart with cardiomyopathy.

The anatomy of the coronary sinus branch vein presents novel problems for cannulation and pacemaker lead insertion. During pacemaker implantation the delivery system must be steerable to properly locate and to be inserted into the appropriate coronary sinus branch vessel. Thereafter, the delivery device must have the ability to be steered through a highly branched vasculature of smaller and smaller vessels, yet it must not be so stiff or biased to be traumatic to the vessels. The delivery system must also provide adequate support within the vessel to avoid kinking, crushing, or otherwise restricting the lumen of the introducer to allow for delivery of the pacemaker lead therethrough. The delivery system must also be extremely flexible at the distal end to navigate the highly tortuous venous anatomy laterally extending from the coronary sinus. After implantation the delivery system must then be able to be removed without displacing the highly flexible, pacemaker lead, which is then disposed in the lumen of the introducer.

Two approaches are commonly used for placement of a medical device within the coronary sinus lateral veins, an inferior approach from below the heart, and a superior approach from above the heart. In the superior approach, the device is advanced through either the right or left cephalic or right or left subclavian vein through the superior vena cava into the right atrium until it is directed toward the coronary sinus. In the inferior approach the device is generally advanced through the femoral vein through the inferior vena cava into the right atrium The tip of the device is then directed toward the ostium of the coronary sinus and subsequently a lateral vein extending from the coronary sinus. The superior approach is the preferred approach and is the approach for which the introducer of the present invention is optimized.

With conventional non-splittable and non-sliceable introducers, the maximum diameter of the pacemaker lead that can be inserted is no larger than the lumen of the introducer. This limitation created a significant problem because of the nature of pacemaker leads. Frequently, the pacemaker lead's proximal end includes an electrical connector for connection to the pulse generator. Because the size of the connecter is often larger than the diameter of the lumen of conventional cardiac introducers or sheaths, standard practice for implantable cardiac pacemakers and defibrillator lead placement are introducers or sheaths that are separable, splittable, sliceable or tearable to assist in the insertion of these electrode leads. Once the introducer directs the placement of the medical device, such as an electrode lead, into the body, the separable, splittable, sliceable or tearable introducer is separated lengthwise as it is withdrawn from die body over the electrical connector of the pacemaker lead. By being separable by sonic means, the size of the lumen of the splittable, sliceable or tearable introducer can remain relatively small as it need be no larger than is necessary for passage of the distal tip of the medical device through the lumen of the introducer. In addition, the prior art uses a splittable hemostatic valve, such as shown in U.S. Pat. Nos. 5,125,904 and 5,312,355, which is utilized in combination with a splittable sheath for introduction of a pacemaker electrode into a patient.

It is a matter of preference, but generally speaking, a non-reinforced splittable introducer is much easier to remove from the body than a braided sliceable introducer without disrupting, the implanted pacing or defibrillating lead. This is due to the fact that the material of the splittable sheath has much less structural integrity than a reinforced catheter, and the act of splitting is more constant and less jerky than when slicing a braided sliceable introducer. When considerable time has been spent in placement of the lead tip in an acceptable anatomical and physiological position, having the lead tip displaced when removing the introducer is extremely disruptive to the procedure and can even result in suboptimal lead positioning. This being said, the majority of physicians would prefer to remove a splittable introducer over a sliceable introducer following lead placement.

Lateral vein introducers are well known and applied to a variety of operations. These introducers consist of a braided sheath (guide) construction for torqueability and support, and have sufficient flexibility to be manipulated around irregular paths, such as occur within the coronary sinus branch vessels. The introducers by themselves are not capable of negotiating the branch vessels of the coronary sinus, but instead rely on the navigation of a guidewire to control the direction for subsequent additional braided guide catheters to follow. It is desirable to have both a radially flexible introducer sheath, braided catheter, and guidewire to avoid trauma to the vessel walls and to more easily track in a tortuous or highly branched venous system, while at the same time to be shaped to minimize the potential for kinking or crushing when traversing acute bends in the vessel. Generally, to be torqueable and radially flexible at the same time meant that the introducer had to include a braided reinforcement in or on it. However, a non-braided introducer sheath, supported by a braided guide has been shown to have equivalent results,

Current non-braided (or non-reinforced) introducer sheaths, however, are unable to be radially flexible enough to atraumatically navigate the tortuous or highly branched venous system without kinking or crushing. Therefore, what is needed is a highly flexible, kink resistant, crush resistant, introducer than can be splittable while being removed from the body.

### BRIEF SUMMARY

The illustrated embodiments of the invention include a cardiac introducer comprising a splittable hemostatic valve, a nontorqueable, splittable proximal sheath portion coupled to and fluidicly communicated to the hemostatic valve, and a, separable distal sheath portion coupled to and fluidicly communicated to the nontorqueable, splittable proximal sheath portion.

The separable distal sheath portion is nonsplittable.

The nonsplittable distal sheath portion is more torqueable than the proximal sheath portion.

The nonsplittable distal sheath portion is biased into a shape adapted for use in a predetermined cardiac application.

The nonsplittable distal sheath portion is reinforced to provide improved integrity and crush resistance.

The reinforcement includes fiber, wire, flat wire, braid, spiral or coil reinforcement, material selection, fillers, or cladding with shrinkable tubing or coatings.

The distal sheath portion sheath comprises a plurality of subportions which are integral and identical in all characteristics with each other or may be comprised of the material identical or similar to the proximal sheath portion, or may assume material characteristics distinct from both other sheath subportions or the proximal sheath portion.

The illustrated embodiments of the invention include a method of using any of the cardiac introducers of any of introducers described above.

The illustrated embodiments of the invention include a method of manufacturing any of the cardiac introducers of any of the introducers described above.

More particularly, the illustrated embodiments of the invention include a cardiac introducer including a splittable hemostatic valve, and a sheath coupled to and fluidicly communicated to the hemostatic valve. The sheath includes a nontorqueable splittable proximal portion coupled to and fluidicly communicated to the hemostatic valve, and a reinforced, non-splittable, sliceable, flexible distal portion coupled to and fluidicly communicated to the nontorqueable, splittable proximal portion.

The reinforced, non-splittable sliceable, flexible distal portion of the sheath is biased into a shape adapted for use in left ventricle access.

The reinforced, non-splittable, sliceable, flexible distal portion of the sheath is soft but crush resistant.

The reinforced, non-splittable, sliceable, flexible distal portion of the sheath is reinforced with fiber, wire flat wire, braid, spiral coil reinforcement, material selection, fillers, or cladding with shrinkable tubing or coatings so that a lumen defined therein remains substantially open.

The reinforced, non-splittable, sliceable, flexible distal portion of the sheath comprises a plurality of subportions which are each comprised of a material the same as or similar to the proximal portion of the sheath.

The reinforced, non-splittable, sliceable, flexible distal portion of the sheath comprises a plurality of subportions each of which are comprised of different materials containing different material characteristics distinct from each other or from the proximal portion of the sheath.

The illustrated embodiments of the invention also include a cardiac introducer includes a splittable hemostatic valve, and a sheath coupled to and fluidicly communicated to the hemostatic valve. The sheath includes a nontorqueable, splittable proximal portion coupled to and fluidicly communicated to the hemostatic valve, and a braid reinforced, splittable, distal portion coupled to and fluidicly communicated to the nontorqueable, splittable proximal sheath portion.

The braid reinforced, splittable, distal portion of the sheath is biased into a shape adapted for use in left ventricle access.

The braid reinforced, splittable, distal portion of the sheath is highly flexible and crush resistant.

The nontorqueable, splittable, proximal portion of the sheath is reinforced.

The reinforcement of the distal and proximal portions of the sheath comprises fiber, wire, flat wire, or formed braid reinforcement.

The braid reinforced, splittable distal portion of the sheath comprises a plurality of subportions which are each comprised of a material the same as or similar to the proximal portion of the sheath.

The braid reinforced, splittable distal portion of the sheath comprises a plurality of subportions which are each comprised of different materials containing different material characteristics distinct from each other or from the proximal portion of the sheath.

The illustrated embodiments still further include a method of using a cardiac introducer including the steps of inserting the cardiac introducer including a sheath into the coronary sinus of a patient, navigating a distal end of the sheath to a desired position, and removing the introducer from the patient by separating at least a portion of the introducer sheath without the aid of any tool and separating another portion of the introducer sheath with the aid of a tool.

The step of removing the introducer from the patient by separating at least a portion of the introducer sheath without the aid of any tool includes peeling apart a proximal portion of the sheath.

The step of removing the introducer from the patient by separating at least a portion of the introducer sheath with the aid of a tool includes slicing apart a distal portion of the sheath.

The step of removing the introducer from the patient by separating at least a portion of the introducer sheath without the aid of any tool includes separating both a proximal and a distal portion of the sheath without the aid of any tool. In such a case either another part of the sheath will be separated with a tool, or the method of using a cardiac introducer includes the steps of inserting the cardiac introducer including a sheath into the coronary sinus of a patient, navigating a distal end of the sheath to a desired position, and removing the introducer from the patient by separating the entire introducer sheath without the aid of any tool, namely both the proximal and distal portions, there being no portion that is to be separated with the aid of a tool.

The step of separating another portion of the introducer sheath with the aid of a tool includes cutting the distal portion of the sheath with an introducer cutting knife.

The step of navigating a distal end of the sheath to a desired position includes accessing the left ventricle of the heart.

The step of navigating a distal end of the sheath to a desired position includes torquing the distal end of the sheath through the coronary sinus without restricting the lumen of the introducer sheath.

While the apparatus and method has or will be described for the sake of grammatical fluidity with functional explanations, it is to be expressly understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112. The disclosure can be better visualized by turning now to the following drawings wherein like elements are referenced by like numerals.

### BRIEF DESCRIPTION OF THE DRAWINGS

FIG. 1 is a top view of the hybrid lateral vein introducer of the illustrated embodiment.

FIG. 2 is a side view of a conventional cutter with a partially split portion of the proximal sheath used in the illustrated embodiment,

FIG. 3 is a top view of the embodiment of the splittable hybrid lateral vein introducer seen in FIG. 1 wherein a portion of the length of the sheath is provided with a peelable means.

FIG. 4 is a top view of an alternative embodiment of the splittable hybrid lateral vein introducer seen in FIG. 1 wherein the entire length of the sheath is provided with a peelable means.

The disclosure and its various embodiments can now be better understood by turning to the following detailed description of the preferred embodiments which are presented as illustrated examples of the embodiments defined in the claims. It is expressly understood that the embodiments as defined by the claims may be broader than the illustrated embodiments described below.

### DETAILED DESCRIPTION OF THE PREFERRED EMBODIMENTS

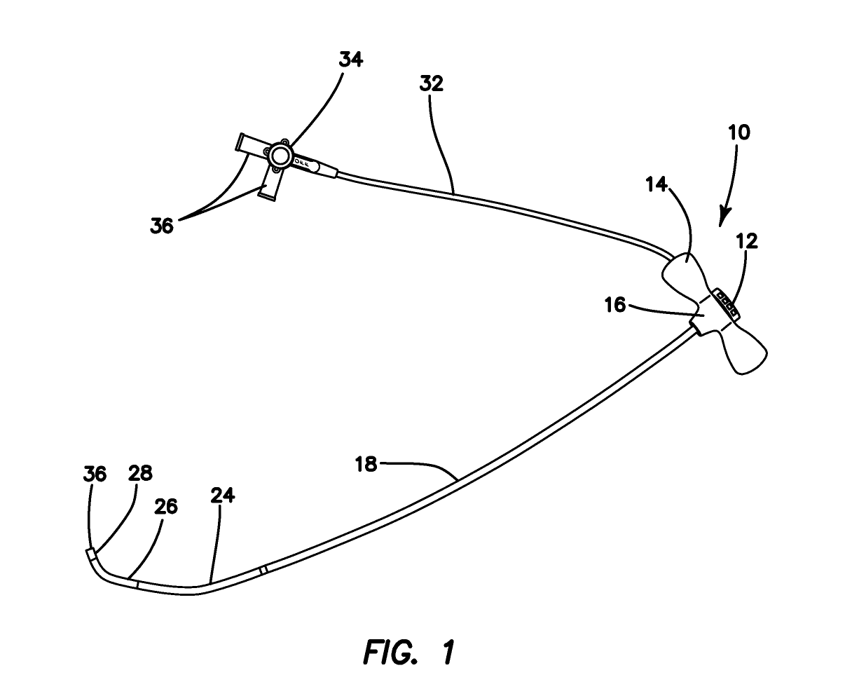

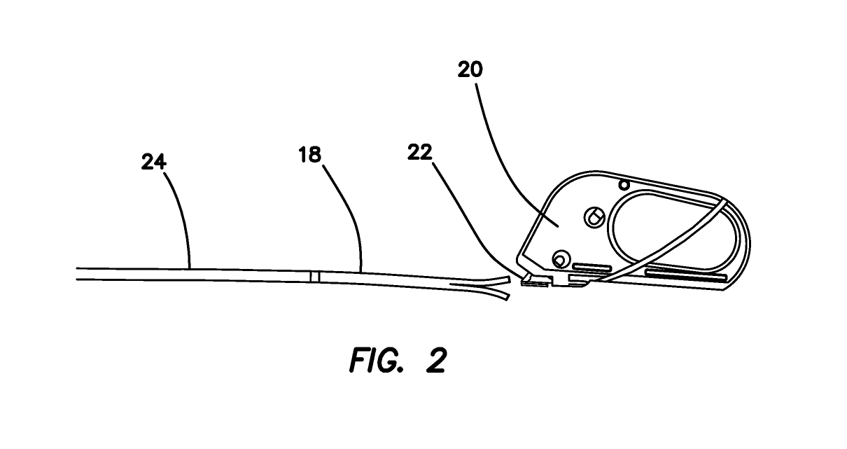

FIG. 1 is a top view of the hybrid lateral vein introducer of the illustrated embodiment. The introducer 10 includes a conventional splittable hemostatic valve 12, which in the illustrated embodiment shows two symmetrical wings 14 extending from a splittable hub 16. A conventional splittable or separable proximal sheath portion 18 is coupled to and extends from hub 16. Introducer 10 may be separable or splittable in any way now know or later devised. The means for rending all or part of the introducer sheath separable or splittable include any means known for splitting, peeling or peeling away a sheath such as score lines, slits, skives, extruded scores, extruded microlumens, molded relief channels, molecularly aligned lines of weakness, oriented material such as PTFE, mechanically or adhesively connected separable or separated portions, thermally separable connected portions or any mechanism that allows portions to be connected and then to be separated without the use of a cutter or other tool. Sliceable for the purposes of this specification will include any extruded, molded, or otherwise formed sheath or hub that is only separable with a cutter, slitter, or other tool. In the illustrated embodiment sheath portion 18 is separable using a conventional introducer cutting knife 20 seen in FIG. 2 with a cutting blade 22 specially shape to lit into sheath portion 18 and to simultaneously make a longitudinal cut into sheath portion 18 as knife 20 is drawn or pushed down the longitudinal length of sheath portion 18.

Introducer 10 in the illustrated embodiment is provided with a flushing line 32 fluidicly communicated to valve 12 and a flushing, stop valve 34 to open or close the flushing port (the output of valve 34), or to fluidicly communicate flushing line 32 to one or one of two or more access ports 36.

In the illustrated embodiment sheath portion 18 is a conventional soft pliable sheath, which has limited torqueability, but adequate flexibility to be maneuvered in the cardiac vascular system. At a predetermined distance from valve 12, sheath 18 is coupled to a reinforced sheath portion 24. Sheath portion 24 is sufficiently reinforced to allow for good integrity and lumen crush resistance while maintaining flexibility of sheath portion 24. The reinforcement may be implemented by any means or measure now known or later devised, including without limitation fiber, wire, flat wire, braid, spiral coil reinforcement, material selection, fillers, or cladding with shrinkable tubing or coatings. Sheath portion 24 extends via sheath portion 26 and tip portion 28 to the distal tip 30 of introducer 10. Sheath portion 26 and tip portion 28 may be integral and identical in all characteristics with sheath portion 24 or may be comprised of the material identical or similar to sheath portion 18, or may assume material characteristics distinct from both sheath portions 18 and 24.

In the illustrated embodiment, sheath portions 24, 26 and 28 are biased or shaped for a particular cardiac application, namely left ventricle access. However, other shapes and other cardiac applications may be accommodated by conventional design principles and still be within the scope of the invention. In the illustrated embodiment portions 24, 26 and 28 are composed of material of increasing durometer respectively. For example an embodiment is also contemplated where a proximal portion of sheath portion 18 is reinforced for torqueability similar to sheath portion 24, and extends to an intermediate portion along, sheath portion 18 where it is coupled to a non-reinforced portion for the remainder of sheath portion 18, where it is coupled to sheath portion 24. The benefit of this embodiment is that the most proximal sheath portion will have additional integrity and torqueability.

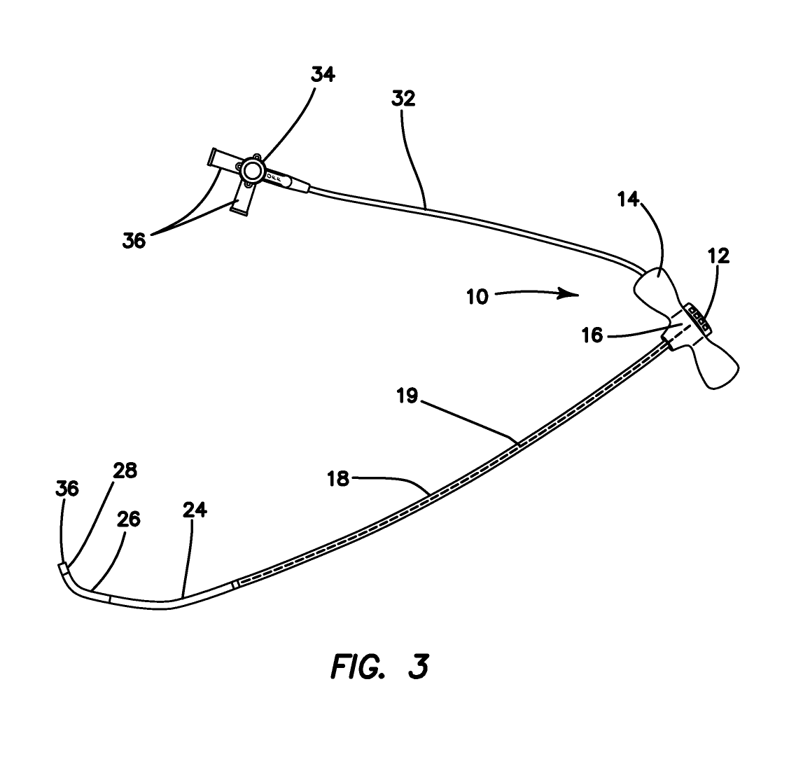

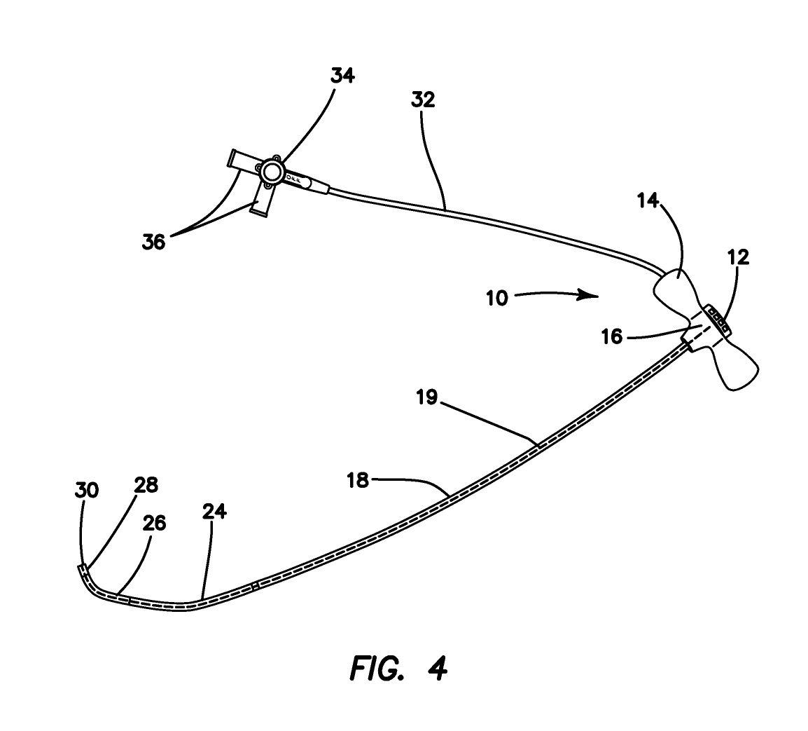

FIG. 3 is a top view of the splittable hybrid lateral vein introducer 10 of the illustrated embodiment in which a proximal segment 19 of the sheath portion 18 is reinforced, but still splittable or separable. In the illustrated embodiment a proximal segment 19 of the sheath portion 18 is reinforced by braiding or other means known or disclosed above and then split, sliced or cut along its entire length during manufacture to form two tubular halves or semi-cylinders. The two semi-cylinders are then placed hack together and heated to fuse or otherwise bonded together. The polymer material of the sheath 18 softens and bonds with a thin connecting layer between the two semi-cylindrical, braid reinforced halves. There are many other ways in which the same structure can be made and it is to be understood that the invention is not to be limited by the disclosed manufacturing method. The resulting sheath 18 is easily splittable or separable on the two opposing lines of separation formed by the fused interconnecting layer of polymer material without the aid of any tool, while still having braided or reinforced halves. In the embodiment of FIG. 3, only the proximal segment 19 of the sheath portion 18 is splittable and the distal portions 24, 26 and 28 are reinforced and may be sliceable with the aid of a knife 20 or scalpel (not shown). In the alternative embodiment seen in FIG. 4, the entire length of the sheath portion 18 including portions 19, 24, 26 and 28 are rendered splittable by the above manufacturing method or other known manufacturing methods.

Many alterations and modifications may be made by those having ordinary skill in the art without departing from the spirit and scope of the embodiments. Therefore, it must be understood that the illustrated embodiment has been set forth only for the purposes of example and that it should not be taken as limiting the embodiments as defined by the following embodiments and its various embodiments.

Therefore, it must be understood that the illustrated embodiment has been set forth only for the purposes of example and that it should not be taken as limiting the embodiments as defined by the following claims. For example, notwithstanding the fact that the elements of a claim are set forth below in a certain combination, it must be expressly understood that the embodiments includes other combinations of fewer, more or different elements, which are disclosed in above even when not initially claimed in such combinations. A teaching that two elements are combined in a claimed combination is further to be understood as also allowing for a claimed combination in which the two elements are not combined with each other, but may be used alone or combined in other combinations. The excision of any disclosed element of the embodiments is explicitly contemplated as within the scope of the embodiments.

The words used in this specification to describe the various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic, to all possible meanings supported by the specification and by the word itself.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such. It is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptionally equivalent, what can be obviously substituted and also what essentially incorporates the essential idea of the embodiments.

We claim:

1. A cardiac introducer comprising:
a splittable hemostatic valve; and
a sheath coupled to and fluidicly communicated to the hemostatic valve, wherein the sheath further comprises:
a nontorqueable, splittable proximal portion coupled to and fluidicly communicated to the hemostatic valve; and
a reinforced, non-splittable, sliceable, flexible distal portion coupled to and fluidicly communicated to the nontorqueable, splittable proximal portion.

2. The cardiac introducer of claim 1 were the reinforced, non-splittable sliceable, flexible distal portion of the sheath is biased into a shape adapted for use in left ventricle access.

3. The cardiac introducer of claim 1 where the reinforced, non-splittable, sliceable, flexible distal portion of the sheath is soft but crush resistant.

4. The cardiac introducer of claim 1 where the reinforced, non-splittable, sliceable, flexible distal portion of the sheath is reinforced with fiber, wire, flat wire, braid, spiral coil reinforcement, material selection, fillers, or cladding with shrinkable tubing or coatings so that a lumen defined therein remains substantially open.

5. The cardiac introducer of claim 1 where the reinforced, non-splittable, sliceable, flexible distal portion of the sheath comprises a plurality of subportions which are each comprised of a material the same as or similar to the proximal portion of the sheath.

6. The cardiac introducer of claim 1 where the reinforced, non-splittable, sliceable, flexible distal portion of the sheath comprises a plurality of subportions each of which are comprised of different materials contain inc different material characteristics distinct from each other or from the proximal portion of the sheath.

7. A cardiac introducer comprising:
a splittable hemostatic valve; and
a sheath coupled to and fluidicly communicated to the hemostatic valve, wherein the sheath further comprises:
a nontorqueable, splittable proximal portion coupled to and fluidicly communicated to the hemostatic valve; and
a braid reinforced, splittable, distal portion coupled to and fluidicly communicated to the nontorqueable, splittable proximal sheath portion.

8. The cardiac introducer of claim 7 where the braid reinforced, splittable, distal portion of the sheath is biased into a shape adapted for use in left ventricle access.

9. The cardiac introducer of claim 7 where the braid reinforced, splittable, distal portion of the sheath is highly flexible and crush resistant.

10. The cardiac introducer of claim 7 where the nontorqueable, splittable, proximal portion of the sheath is reinforced.

11. The cardiac introducer of claim 10 where the reinforcement of the distal and proximal portions of the sheath comprises fiber, wire, flat wire, or formed braid reinforcement.

12. The cardiac introducer of claim 7 where the braid reinforced, splittable distal portion of the sheath comprises a plurality of subportions which are each comprised of a material the same as or similar to the proximal portion of the sheath.

13. The cardiac introducer of claim 7 where the braid reinforced, splittable distal portion of the sheath comprises a plurality of subportions which are each comprised of different materials containing different material characteristics distinct from each other or from the proximal portion of the sheath.

14. A method of using a cardiac introducer comprising:
inserting the cardiac introducer comprising a sheath into the coronary sinus of a patient;
navigating a distal end of the sheath to a desired position; and
removing the introducer from the patient by separating, at least a portion of the introducer sheath without the aid of any tool and separating another portion of the introducer sheath with the aid of a tool.

15. The method of claim 14 where removing the introducer from the patient by separating at least a portion of the introducer sheath without the aid of any tool comprises peeling apart a proximal portion of the sheath.

16. The method of claim 15 where removing the introducer from the patient by separating at least a portion of the introducer sheath with the aid of a tool comprises slicing apart a distal portion of the sheath.

17. The method of claim 14 where removing the introducer from the patient by separating at least a portion of the introducer sheath without the aid of any tool comprises separating both a proximal and a distal portion of the sheath without the aid of any tool.

18. The method of claim 16 where separating another portion of the introducer sheath with the aid of a tool comprises cutting the distal portion of the sheath with an introducer cutting knife.

19. The method of claim 14 where navigating a distal end of the sheath to a desired position comprises accessing the left ventricle of the heart.

20. The method of claim 14 where navigating a distal end of the sheath to a desired position comprises torquing the distal end of the sheath through the coronary sinus without restricting the lumen of the introducer sheath.
